(19) United States
(12) Patent Application Publication
(10) Pub. No.: US 2005/0119172 A1
(43) Pub. Date: Jun. 2, 2005

(54) Stabilization of protein preparations

(76) Inventor: Werner Merkle, Nottingham (GB)

Correspondence Address: NIXON & VANDERHYE, PC, 1100 N GLEBE ROAD, 8TH FLOOR, ARLINGTON, VA, 22201-4714, US

(21) Appl. No.: 10/503,520

(22) PCT Filed: Feb. 5, 2003
(86) PCT No.: PCT/GB03/00474

(30) Foreign Application Priority Data
Feb. 5, 2002 (GB) 0202633.4

(51) Int. Cl. A61K 38/38

(52) U.S. Cl. 514/12

(57) ABSTRACT

A composition comprising a non-albumin protein is stabilised by the addition of a highly purified recombinant human serum albumin. The non-albumin protein may be Factor VIII.

### FIELD OF THE INVENTION

This invention relates to the stabilization of compositions containing proteins, in particular to the use of highly purified recombinant human serum albumin (rHA) to stabilize compositions containing proteins, particularly proteins obtained by recombinant methods. In particular embodiments, the invention relates to the use of highly purified rHA to stabilize compositions containing recombinant Factor VIII (rF-VIII).

### BACKGROUND OF THE INVENTION

Factor VIII (F-VIII) is a plasma protein that is involved in, and is essential to, the blood clotting process. Deficiency in F-VIII leads to the congenital bleeding disorder Haemophilia A. Patients with gross deficiencies of F-VIII (eg of the order of 1-2% of normal levels) may suffer from spontaneous bleeding and severe bleeding following trauma. Such bleeding into enclosed areas of the body is a major cause of morbidity in such patients.

Compositions comprising F-VIII are characterised in terms of the F-VIII “activity”, expressed in terms of International Units (IU). One IU, as defined by the World Health Organization standard for blood coagulation F-VIII, human, is approximately equal to the level of F-VIII activity found in 1 ml of fresh pooled human plasma). A clinician will prescribe the administration of a certain number of IU to a patient and it is therefore clearly desirable for the indicated F-VIII activity of a composition to be a reliable indicator of the true F-VIII activity. In other words, it is desirable for the F-VIII activity to be constant, and not to change between the time of manufacture of the composition and the time of administration to the patient.

Effective treatment of Haemophilia A involves replacement of the missing Factor VIII clotting factor by infusion, either in response to bleeding or in a regular prophylactic administration scheme. Replacement Factor VIII was originally obtained by isolation from human plasma. However, more recently attempts have been made to produce rF-VIII. Factor VIII from recombinant sources may be beneficial in that it avoids potential contaminants that may be present in blood products, as well not being subject to potential limitations on supply associated with material isolated from donated blood.

Clearly, since a major benefit of rF-VIII is that it is free of potential contaminants that might be present in material isolated from blood, it is desirable for any composition in which the rF-VIII is formulated to be similarly free of material of blood origin. However, it is found in practice that rF-VIII is somewhat unstable and has a limited shelf-life. In attempts to overcome this problem, serum-derived albumin has been incorporated in rF-VIII compositions to act as a stabiliser, but this reintroduces a risk of contamination. Albumin-free formulations have also been proposed, with additional materials such as various ionic salts, sugars and amino acids as stabilisers. However, in order to achieve satisfactory stabilisation of the formulation these additional excipients may need to be present in rather high concentrations, and may suffer from other disadvantages such as unsuitability for lyophilisation and reconstitution.

Similar comments apply not only to the stabilisation of formulations of rF-VIII but to formulations of recombinant proteins in general.

rHA has also been proposed as a stabiliser for protein compositions, but to date no formulation of rF-VIII has been developed which is satisfactory for commercial use and which contains rHA as stabiliser.

### SUMMARY OF THE INVENTION

It has now been found that highly purified rHA is effective in stabilising formulations of proteins, especially recombinant proteins, and in particular rF-VIII, and overcomes or substantially mitigates some or all of the above-mentioned or other disadvantages or shortcomings of the prior art.

According to a first aspect of the invention, there is provided a composition comprising a non-albumin protein, the composition further comprising a highly purified rHA in an amount sufficient to stabilise the protein.

The incorporation of the highly purified rHA into compositions comprising non-albumin protein may stabilise the compositions. In particular, the highly purified rHA may inhibit or prevent modification or degradation of the non-albumin protein over time (many such proteins are labile and can become unstable when stored for protracted periods). The inclusion of highly purified rHA may lead to satisfactory stability of the composition, even at relatively low concentrations of rHA. The use of highly purified rHA has also been found to preserve the activity of the non-albumin protein, eg F-VIII.

The invention is particularly useful in relation to the stabilisation of compositions comprising non-albumin protein prepared by recombinant methods.

The rHA may also be incorporated into formulations comprising other materials known to exert a stabilising effect on recombinant proteins. In such cases, the presence of the rHA may result in lower, sometimes considerably lower, concentrations of such other materials being required in order to achieve satisfactory stability.

Thus, according to a second aspect of the invention, there is provided a composition comprising a non-albumin protein, the composition further comprising highly purified rHA and one or more additional stabilising agents.

Such additional stabilising agents may be selected from:
ionic salts, notably chlorides such as potassium chloride, sodium chloride and calcium chloride;
amino acids, eg histidine, lysine, glycine, arginine, etc;
sugars, eg mannitol, sucrose, fructose, lactose and maltose;
detergents, notably non-ionic detergents, and especially polyoxyethylene sorbitan esters (eg polysorbates);
polymers, notably synthetic polymers, and especially hydrophilic synthetic polymers, eg polyethylene glycols.

The highly purified rHA used in the invention, while necessarily present in the final composition, may not be required during processing stages leading to the final composition. For instance, during fermentation and/or purification of a recombinant non-albumin protein, rHA may be used for stabilisation, but that rHA may not necessarily be highly purified rHA.

Thus, according to a third aspect of the invention, there is provided a process for the preparation of a composition comprising a non-albumin recombinant protein, which process comprises the steps of
a) causing a cell transformed with a nucleotide sequence coding the non-albumin recombinant protein to express the non-albumin recombinant protein; and
b) isolating and/or purifying the non-albumin recombinant protein;
   wherein step a) and/or step b) is carried out in the presence of a first form of rHA, which first form of rHA is less pure than a second form of rHA;
c) separating the isolated and/or purified non-albumin protein obtained in step b) from the first form of rHA; and
d) combining the isolated and/or purified non-albumin recombinant protein with the second form of rHA and optionally with other excipients in order to provide a stable composition.

As noted above, one of the benefits of the present invention is that the incorporation of highly purified rHA in compositions comprising F-VIII has been found to preserve the activity of the F-VIII.

Thus, according to a fourth aspect of the invention, there is provided a method for preserving or maintaining the F-VIII activity of a composition comprising F-VIII, particularly rF-VIII, which method comprises adding to the composition a stabilising amount of highly purified rHA.

### DETAILED DESCRIPTION OF THE INVENTION

The composition according to the invention is normally used in the form of an aqueous solution or suspension. However, the composition may be made up to such a form only immediately or shortly prior to use. The composition may be supplied and stored in a powdered form, eg a powder prepared by lyophilisation, reconstitution with water being carried out prior to administration of the composition to a patient. Such procedures are conventional in this field, and will be familiar to those skilled in the art.

Typically, the composition is supplied in the form of a lyophilised (freeze-dried) powder, in a sealed vial. The composition is reconstituted with a specified volume of water for injection, most commonly 2.5, 5 or 10 ml of water.

When reconstituted with water, the composition according to the first or the second aspect of the invention will typically comprise from about 0.1 mg/ml up to about 20 mg/ml highly purified rHA, more commonly up to about 10 mg/ml, and may typically comprise up to about 7 mg/ml, or up to about 5 mg/ml, or up to about 1 mg/ml highly purified rHA.

In addition to the non-albumin protein (eg rF-VIII) and the highly purified rHA, the composition may optionally contain one or more of a number of further stabilising agents and other excipients.

Further stabilising agents that may be included are, as noted above, ionic salts, amino acids, sugars, detergents and polymers.

Where ionic salts are present, it is particularly preferred that the salts should be selected from potassium chloride, sodium chloride and calcium chloride. In such a case, the concentration of chloride ion, following reconstitution with water, may be in the range 0.05 to 2 mg/ml.

Where amino acids are present, it is particularly preferred that the amino acid should be one or more of histidine, lysine, glycine and arginine. The overall concentration of amino acid(s) in the reconstituted composition may vary over a wide range, eg from 0.1 to 100 mg/ml, more preferably 0.1 to 50 mg/ml, and may be considerably lower, eg the concentration may be less than 10 mg/ml, eg in the range 0.01 to 10 mg/ml.

Where detergents are present, non-ionic detergents are preferred, and in particular, polyoxyethylene sorbitan esters (polysorbates). Polyoxyethylene (20) sorbitan monooleate (Polysorbate 80) is particularly preferred. The concentration of detergent in the reconstituted composition may be very low, eg less than 1 mg/ml, more preferably less than 0.1 mg/ml, eg 0.001 to 0.1 mg/ml.

Synthetic polymers, especially hydrophilic synthetic polymers, may also be incorporated into the composition. A preferred class of synthetic polymer is polyethylene glycol. The polymer preferably has an average molecular weight of less than 10,000 daltons, more preferably less than 5,000 daltons. Where a synthetic polymer is present in the reconstituted composition, its concentration may be from 0.1 to 10 mg/ml, or lower, eg the concentration may be less than 1 mg/ml, eg 0.01 to 1 mg/ml.

Where sugars are included in the composition, they may be selected from mannitol, sucrose, fructose, lactose and maltose. The concentration of sugar in the reconstituted composition may be in the range 1 to 50 mg/ml, more preferably 1 to 20 mg/ml or 5 to 15 mg/ml, but may be considerably lower, eg 0.1 to 5 mg/ml.

Other excipients that may be present in the composition include buffering agents, for example salts of citric acid, eg sodium citrate.

Since the additional excipients and/or further stabilising agents referred to above are optional, it will be appreciated that the concentration of such agents in the reconstituted composition may be less than the lower limits quoted above, and may be zero (or effectively zero, by which is meant below detectable limits). Thus, the concentration may be from zero to the upper, preferred limits quoted above (or higher).

It will also be appreciated that other excipients may be introduced into the composition, generally at low levels, by virtue of being present in, for instance, the rHA used in the composition. Preparations of rHA may contain, for instance, certain amounts of octanoic acid (or a salt thereof), N-acetyltryptophan or detergent such as Polysorbate 80. Such additional excipients are generally present at rather low levels and their concentration in the final composition (in which the concentration of rHA may only be of the order of 0.5%) will be correspondingly lower again.

Expression of the non-albumin recombinant protein in the third aspect of the invention (or for use in the compositions of the second or third aspects) may be brought about by methods that will be familiar to those skilled in the art. The recombinant cells may be eukaryotic or prokaryotic. The recombinant cells may be bacteria (for example Escherichia coli or Bacillus subtilis), yeasts (for example a yeast of the genus Saccharomyces (eg S. cerevisiae), the genus Kluyveromyces (eg K. lactis) or the genus Pichia (eg P. pastoris)), filamentous fungi (for example Aspergillus), plants or plant cells, animals or animal cells (which may be transgenic) or insect cells.

In a preferred embodiment, the non-albumin recombinant protein may be derived from a fungal culture medium obtained by culturing a fungus transformed with an appropriate encoding nucleotide sequence in a fermentation medium, whereby said fungus expresses the protein and secretes it into the medium. The fungus may be a filamentous fungus such as an Aspergillus species. Preferably, the fungus is a yeast. More preferably the fungus is of the genus Saccharomyces (eg S. cerevisiae), the genus Kluyveromyces (eg K. lactis) or the genus Pichia (eg P. pastoris).

Isolation, purification and separation of the non-albumin recombinant protein may also be carried by techniques that are known per se to those skilled in the art.

Although described above with particular reference to rF-VIII, other proteins may be stabilised using highly purified rHA in accordance with the invention. Examples of such recombinant proteins include all or part of an enzyme, an enzyme inhibitor, an antigen, an antibody, a hormone, a factor involved in the control of coagulation, an interferon, a cytokine [the interleukins, but also their variants which are natural antagonists of their binding to the receptor(s), the SIS (small induced secreted)-type cytokines and for example the macrophage inflammatory proteins (MIPs), and the like], of a growth factor and/or a factor involved in cell differentiation [and for example the transformant growth factors (TGFs), the blood cell differentiation factors (erythropoietin, M-CSF, G-CSF, GM-CSF and the like), insulin and the growth factors resembling it (IGFs), or alternatively cell permeability factors (VPFNEGF), and the like], of a factor involved in the genesis/resorption of bone tissues (OIF and osteospontin for example), of a factor involved in cellular motility or migration [and for example autocrine motility factor (AMF), migration stimulating factor (MSF), or alternatively the scatter factor (scatter factor/hepatocyte growth factor)], of a bactericidal or antifungal factor, of a chemotactic factor [and for example platelet factor 4 (PF4), or alternatively the monocyte chemoattracting peptides (MCP/MCAF) or neutrophil chemoattracting peptides (NCAF), and the like], of a cytostatic factor (and for example the proteins which bind to galactotsides), of a plasma (and for example von Willebrand factor, fibrinogen and the like) or interstitial (laminin, tenascin, vitronectin and the like) adhesive molecule or proteins involved in formation of extracellular matrices, or alternatively any peptide sequence which is an antagonist or agonist of molecular and/or intercellular interactions involved in the pathologies of the circulatory and interstitial compartments and for example the formation of arterial and venous thrombi, cancerous metastases, tumour angiogenesis, inflammatory shock, autoimmune diseases, bone and osteoarticular pathologies and the like.

The non-albumin protein used in the invention is most preferably recombinantly produced. Thus, a polynucleotide encoding the protein is transformed into a cell and expressed. Many expression systems are known, including bacteria, yeasts, filamentous fungi, plant cells, animal cells and insect cells.

Sources of rHA

Processes for the preparation of rHA will in general be familiary to those skilled in the art and are described, for instance, in WO 96/37515 and WO 00/44772.

In a preferred embodiment of the present invention an initial rHA solution is derived from a fungal culture medium obtained by culturing a fungus transformed with an rHA-encoding nucleotide sequence in a fermentation medium, whereby said fungus expresses rHA and secretes it into the medium. The fungus may be a filamentous fungus such as an Aspergillus species. Preferably, the fungus is a yeast. More preferably the fungus is of the genus Saccharomyces (eg S. cerevisiae), the genus Kluyveromyces (eg K. lactis) or the genus Pichia (eg P. pastoris).

Preferably, at least some of the rHA is produced by a cell which comprises a recombinant albumin coding sequence wherein the 3′ end of the recombinant albumin coding sequence comprises two or more in-frame translation stop codons, and preferably three in-frame translation stop codons, or by a process comprising culturing a fungal cell expressing a recombinant albumin coding sequence and obtaining the rHA, wherein the cell has a genetic modification which causes the cell to have at least a reduced capacity of mannosylation of the recombinantly-expressed albumin and wherein the culture medium is at least 1,000 litres and is of pH5.5-6.8.

The recombinant cells may be eukaryotic or prokaryotic. The recombinant cells may be bacteria (for example E. coli or Bacillus subtilis), yeasts (for example a yeast of the genus Saccharomyces (eg S. cerevisiae), the genus Kluyveromyces (eg K. lactis) or the genus Pichia (eg P. pastoris)), filamentous fungi (for example Aspergillus), plants or plant cells, animals or animal cells (which may be transgenic) or insect cells.

As used herein, genetic modification preferably means any suppression, substitution, deletion or addition of one or more bases or of a fragment of the fungal cell DNA sequences. Such genetic modifications may be obtained in vitro (directly on isolated DNA) or in situ, for example by genetic engineering techniques or by exposing the fungal cells to mutagenic agents. Mutagenic agents include for example physical agents such as energetic rays (X-rays, y-rays, UV, etc) or chemical agents capable of reacting with different functional groups of DNA, such as alkylating agents (ethyl methanesulphonate (EMS), 4-nitroquinoline N-oxide (NQO), etc), bisalkylating agents, intercalating agents, etc. Genetic modifications may also be obtained by genetic disruption, for example according to the method disclosed by Rothstein et al. [Meth. Enzymol. 194 (1991), 281-301]. According to this method, part or all of a gene is replaced, through homologous recombination, by an in vitro modified version. Genetic modifications can also be obtained by any mutational insertion on DNA sequences, such as transposons, phages, etc.

It is known that certain modifications such as point mutations can be reversed or attenuated by cellular mechanisms. Such modifications may not provide the most useful forms of modified fungal cells since their phenotypic properties may not be very stable. Accordingly, it is preferred that the genetic modification(s) are stably inherited and/or are non-reverting and/or are non-leaky. Such modification(s) are generally obtained by a deletion or a gene disruption.

By a “leaky mutant” and grammatical variants thereof, we include mutants that result from a partial rather than a complete inactivation of the wild-type function.

The genetic modifications carried by the fungal cells may be located in a coding region of the DNA sequences of the cell and/or in a region affecting the expression of a gene. More particularly, said modifications will generally affect the coding region or the region responsible for or involved in the expression of one or more genes whose expression products are enzymes involved in mannosylation.

The reduced capacity of the fungal cells to mannosylate proteins may therefore result from the production of inactive enzymes due to structural and/or conformational changes, from the production of enzymes having altered biological properties, from the absence of production of said enzymes, or from the production of said enzymes at low levels.

The fungal cell mannosylation pathway involves attachment of a first mannosyl residue to the hydroxyl group of seryl and/or threonyl amino acids of proteins or peptides, and then the extension to O-linked di- and oligosaccharides by subsequent addition of mannosyl residues. The first mannosyl residue is transferred from dolichol monophosphate mannose (Dol-P-Man) to the protein in the endoplasmic reticulum, and the additional mannosyl residues are transferred from GPD-Man in the golgi.

In a preferred embodiment, the modified fungal cells carry genetic modifications in at least one gene whose expression product is involved in the attachment of a mannosyl residue to the hydroxyl group of seryl or threonyl amino acids.

In another preferred embodiment, the modified fungal cells carry genetic modifications in at least one gene whose expression product is involved in the transfer of a mannosyl residue from the Dol-P-Man precursor to the hydroxyl group of seryl or threonyl amino acids. Still more preferably, one of these genes is a PMT gene (eg PMT1, PMT2, PMT3, PMT4, PMT5, PMT6 or PMT7). Preferably the PMT gene is PMT1, PMT5 or PMT7.

A growth medium of pH6.0-6.8 is beneficial in terms of host cell integrity during large scale fermentation.

In addition to modifications in a gene involved in the attachment of mannosyl residues to the hydroxyl group of seryl or threonyl amino acids, fungal cells may also carry modifications in the genes involved in subsequent additions of mannosyl residues leading to di- or oligosaccharides, or in the synthesis of the mannosyl residues donor (Dol-P-Man).

Preferably, the fungal cell has a genetic modification within a PMT gene or a gene which affects the expression or product of a PMT gene. A gene which affects the expression of a PMT gene may, for example, affect mRNA transcript levels or PMT product levels.

The fungal cell can be chosen from filamentous fungi and yeasts. Preferably, the cells are yeasts, for example a yeast of the genus Saccharomyces (eg S. cerevisiae), the genus Kluyveromyces (eg K. lactis) or the genus Pichia (eg P. pastoris).

Preferably, the fungal cell expressing the recombinant albumin coding sequence is cultured in a culture medium of at least 5,000 litres, more preferably at least 7,500 litres.

In one embodiment, the fungal cell expressing the recombinant albumin coding sequence is cultured in a culture medium which is maintained in the range of pH6.2-6.7, more preferably pH6.3-6.5. Preferably, the pH of the culture medium is maintained using a pH controller set at a pH between pH6.3 and pH6.5, preferably at a pH between 6.35 and 6.45 and more preferably at about pH6.4. Preferably, the pH controller is controlled within 0.20 or 0.10 pH units of any pH value within any one of the aforementioned pH ranges or within 0.20 or 0.10 pH units of pH6.4.

In an alternative embodiment, the fungal cell is cultured in a culture medium which is maintained in the range of pH5.30-pH5.90, preferably pH5.50-pH5.90, pH5.40-pH5.90 or pH5.40-5.60. Preferably, the lower control set point is between pH5.40 and pH5.60, preferably between pH5.45 and pH5.55, and preferably the lower control set point is about pH5.50.

Characteristics of Highly Purified rHA

Highly purified rHA of use in accordance with the invention may exhibit one or more of the following properties:

(i) extremely low levels of colourants. The term “colourant” as used herein means any compound which colours albumin. For example, a pigment is a colourant which arises from the organism, such as yeast, which is used to prepare recombinant albumin, whereas a dye is a colourant which arises from chromatographic steps to purify the albumin.

(ii) extremely low levels of, or be essentially free of, aluminium, lactate, citrate, metals, non-albumin human proteins, such as immunoglobulins, pre-kallikrein activator, transferrin, α₁-acid glycoprotein, haemoglobin and blood clotting factors, prokaryotic proteins, fragments of albumin, albumin aggregates or polymers, or endotoxin, bilirubin, haem, yeast proteins, animal proteins and viruses. By “essentially free” is meant below detectable levels.

(iii) at least 99.5% monomeric and dimeric, preferably essentially 100% monomeric and dimeric. Up to 0.5%, preferably 0.2% trimer is tolerable but larger forms of albumin are generally absent.

(iv) a nickel ion level of less than 100 ng, based on one gram of albumin, as measured by the method defined in WO 00/44772.

(v) a glycation level of less than 0.6, preferably less than 0.10, 0.075 or 0.05 moles hexose/mole protein as measured in the Amadori product assay, a microassay for glycated protein. The microassay measures the stable Amadori product (AP) form of glycated protein, by oxidation of the C-1 hydroxyl groups of AP with periodate. The formaldehyde released by periodate oxidation is quantitated by conversion to a chromophore, diacetyldihydrolutidine (DDL), by reaction with acetylacetone in ammonia. DDL is then detected colorimetrically. Samples are assayed after desalting using a Pharmacia PD-10 (G25 Sephadex) column and then the albumin in the samples is re-quantitated by the Bradford method (Bradford, 1976, Anal. Biochem., 72, 248-254) and 10 mg albumin assayed. A fructose positive control is included, and the absorbances are read on a Shimadzu UV 2101 spectrophotometer at 412 nm. For every mole of hexose one mole of Amadori product is formed.

(vi) at least 90% or 95%, preferably at least 96%, more preferably at least 97%, even more preferably at least 98%, even more preferably at least 99%, most preferably substantially 100% of the albumin molecules have an intact C-terminus.

(vii) a content of Concanavalin A-binding albumin of less than 0.5% (w/w), preferably less than 0.3%, 0.2% or 0.15%. Concanavalin A (Con A) binds molecules which contain α-D-mannopyranosyl, α-D-glucopyranosyl and sterically related residues. Con A-binding can be assayed using Con A Sepharose (Pharmacia, Cat. No. 17-0440-01) affinity chromatography of rHA in order to determine the content of mannosylated albumin. rHA is diluted to 5% (w/v) with 145 mM sodium chloride then 1:1 with Con A dilution buffer (200 mM sodium acetate, 85 mM sodium chloride, 2 mM magnesium chloride, 2 mM manganese chloride, 2 mM calcium chloride pH5.5). 100 mg rHA is then loaded onto an equilibrated 2 ml Con A Sepharose column which is then washed (5×4 ml) with Con A equilibration buffer (100 mM sodium acetate, 100 mM sodium chloride, 1 mM magnesium chloride, 1 mM manganese chloride, 1 mM calcium chloride pH5.5). The column is eluted with 6 ml Con A elution buffer (100 mM sodium acetate, 100 mM sodium chloride, 0.5M methyl-α-D-mannopyranoside pH5.5). Monomeric albumin in eluate (diluted as appropriate to make sure the sample falls in the middle of the standard curve) is quantified by the Bradford method using a 0-0.12 mg/ml albumin standard curve, and the Con A binding albumin monomer recovered in the eluate is expressed as a percentage of the load.

(viii) a free thiol content of at least 0.85, 0.8, 0.75, 0.7, 0.65 or 0.60 mole SH/mole protein when measured by using Ellman's Reagent, 5,5′-dithiobis-(2-nitrobenzoate) (DTNB), which is a specific means of detecting free sulfhydryl groups such as cys-SH (Cys-residue 34 in the case of rHA). The reaction releases the 5-thio-2-nitrobenzoate ion TNB²⁻ which has an absorption maximum at 412 nm. By measuring the increase in absorbance at 412 nm and dividing by the molar extinction coefficient of the TNB²⁻ ion at 412 nm, the free sulfhydryl content of rHA can be calculated.

(ix) substantially no C18 or C20 fatty acids, when analysed by acidic solvent extraction and gas chromatography of free fatty acids using a C17:0 internal standard.

(x) a high degree of molecular weight homogeneity, specifically a molecular weight distribution of at least 50, 60, 70, 80, 90, 95, 98, 99, 99.9 or substantially 100% of albumin molecules with a molecular weight spread no greater than 2000, 1500, 1000, 900, 800, 700, 600, 500, 400, 300, 200 or fewer daltons when determined by mass analysis using electrospray mass spectrometry. Protein samples are desalted employing standard methods such as dialysis or chromatography and exchanged or diluted into typically 50% (v/v) organic solvent, such as acetonitrile or methanol supplemented with acid, such as 0.1-10% (v/v) formic acid for positive ion electrospray or with base such as 0.1-10% (v/v) ammonium hydroxide for negative ion electrospray. Protein solutions at concentrations optimal for the employed ion source, typically 1-50 μM, are introduced into the electrospray ion source at appropriate flow rates of typically 0.01-100 μl/min using standard methods such as continuous flow, loop injection, or off-line, using a syringe pump, a HPLC pump or nanoelectrospray vial respectively. The instrument analyser/s are tuned for optimal transmission and resolution (the latter should exceed 500, as defined by baseline separation of 500-501 m/z) and calibrated using a local protocol and a suitable calibrant typically a protein (eg horse myoglobin) or a surfactant (eg PEG). Spectra are acquired, averaged and processed to subtract baseline noise, smooth signal, centroid peaks, measure mass and deconvolute data to a true mass scale using appropriate software known in the art and commercially available. One example protocol (Bertucci et al, 2001, Biochimica et Biophysica Acta, 1544, 386-392) utilises dilution in H₂O/CH₃CN 50:50 with 5% HCOOH, to a final albumin concentration of 40-50 μM and analysis by Ionspray Mass Spectrometry, carried out on a Perkin-Elmer SCIEX API III triple quadrupole mass spectrometer (Sciex, Thomill, Canada) equipped with an articulated Ionspray interface, operated using the following parameters: ionspray voltage 5.5 kV; orifice voltage 90 V; scan range 1400-2200 mass units; scan time 8.42 s; resolution>1 mass unit. The spectra are acquired in Multichannel acquisition (MCA) mode summing 20 scans. Analysis is performed by continuous infusion into the source by a Harvard model 22 syringe pump (Harvard Apparatus, South Natick, Mass.) at 5 TI/min flow rate. All measurements are carried out at room temperature. For example, such highly homogeneous HSA may have one of the above mentioned percentages of protein molecules within the range of 66400 to 67000 daltons. Typically, when analysed by one of the above methods, the main peak obtained for a highly homogeneous rHA is usually within 0.05%, more usually within 0.01% of the theoretical mass (in the case of HSA, the theoretical mass is 66,438 daltons). In one embodiment, the mass profile is determined by electrospray mass spectrometry (ESI-MS) with a span of 1000 daltons either side of the main peak. The profile is scrutinised for the presence of micro-heterogeneity observed as discretely resolved components or alternatively as a broadening in the mass peak widths at half height. The relative abundance of resolved components may be measured as relative ion count and expressed as percentage (%) composition. The profile of highly homogeneous rHA as used in the present invention is typically at least 50%, 60%, 70%, 80%, 90% or more similar to native (unmodified) primary structure composition. The relative abundance of components in highly homogeneous rHA may be additionally measured using other quantitative techniques, including neutral coated capillary zone electrophoresis with detection by absorbance in the UV region (Denton & Harris, 1995, J. Chromatog. A., 705, 335-341).

The homogeneity of a population of rHA molecules, may be determined by electrophoretic and chromatographic techniques. For example, SDS PAGE may be performed using standard methods. Local protocols should employ PAGE gels capable of separating proteins within the 20-200 kDa molecular size range. Native PAGE is optimised to yield focusing and separation of proteins with differing mass and/or charge. Electrophoretically separated protein components are visualised by chemical staining methods (eg Coomassie Blue dyes), which should have detection limits of typically greater than 0.1 μg. Quantitation is achieved by subsequent densitometric absorbance scanning with calibration against protein standards.

Gel permeation chromatography is performed using a column, typically with a separation range of 10-500 kDa molecular size, and with analytical dimensions. An optimally buffered aqueous mobile phase is pumped using an HPLC system and eluting components are detected by absorbance in the UV region. Peak quantitation is facilitated by chromatogram integration and calibration against standards of the test rHA.

Usually, when analysed by SDS PAGE, native PAGE and gel permeation chromatography, a highly homogeneous rHA preparation will display one or two of the following features—
(a) at least 99%, preferably 99.9%, of the protein molecules in the population will be rHA.
(b) no more than 10, 9, 8, 7, 6, 5, 4, 3%, preferably no more than 2%, of albumin protein molecules in the population will be dimeric.

The homogeneity of population of rHA molecules, may also be analysed by electrospray mass spectrometry (ESMS) and by peptide mapping.

In a preferred embodiment, when analysed by ESMS and peptide mapping, a highly homogeneous rHA preparation will have the correct native primary sequence of full length HSA or a fragment thereof as defined above and will not have post-translational modifications.

In one embodiment, highly homogeneous rHA has at least two or three of the features (v), (viii), and (x) as defined above. One of the features may be feature (x) in combination with one or two of features (v) and (viii).

In a particularly preferred embodiment, the highly purified rHA is characterized by the following combination of characteristics:
(i) a molecular weight distribution of at least 90, and more preferably 95, 98, 99, 99.9 or substantially 100%, of albumin molecules with a molecular weight spread no greater than 1000 daltons, or more preferably no greater than 900, 800, 700, 600, 500, 400, 300, 200 or fewer daltons, when determined by mass analysis using electrospray mass spectrometry;
(ii) a glycation level of less than 0.10, and more preferably less than 0.075 or 0.05, moles hexose/mole protein; and
(iii) a content of Concanavalin A-binding albumin of less than 0.3% (w/w), and more preferably less than 0.2% or 0.15%.
   rHAcolourantcolourantrHArHArHArHArHA

Highly purified rHA of use in the present invention may be prepared by various processes, including the following:

First Process for Preparing Highly Purified rHA

A first process for preparing highly purified rHA comprises the step of subjecting a first rHA solution of pH8.0-9.5, and having a conductivity in the range of 1 to 75 mS/cm, to an affinity chromatography step which is run in negative mode with respect to the rHA and which utilises an affinity matrix comprising immobilised dihydroxyboryl groups, thereby obtaining a purified rHA solution.

Preferably, the pH of the first rHA solution is pH8.0-9.0, and more preferably pH8.3-pH8.6. It is preferred that the first rHA solution is buffered with a buffer having a pH within the aforementioned pH ranges.

Preferably, the buffer comprises an amino acid at a concentration of 10-500 mM, preferably 25-200 mM, and more preferably 50-150 mM. Preferably the amino acid is glycine.

Preferably, the buffer comprises a monovalent cation at a concentration of 0-500 mM, preferably 25-200 mM, and more preferably 50-150 mM. Preferably, the monovalent cation is sodium, preferably in the form of NaCl. Accordingly, in a preferred embodiment the buffer comprises NaCl at a concentration of 0-500 mM, preferably 25-200 mM, and more preferably 50-150 mM.

Preferably, the buffer comprises a divalent cation at a concentration of 5-250 mM, preferably 10-100 mM. Preferably, the divalent cation is calcium, preferably in the form of CaCl₂. Accordingly, in a preferred embodiment the buffer comprises CaCl₂, at a concentration of 5-250 mM, preferably 10-100 mM.

In a particularly preferred embodiment the first rHA solution and/or buffer comprises about 100 mM glycine, about 100 mM NaCl and about 50 mM CaCl₂.

Preferably, the conductivity of the first rHA solution and/or buffer is 10-50 mS/cm and more preferably 18-22 mS/cm.

Advantageously, the concentration of the rHA in the first rHA solution is in the range of 20-120 g/l, preferably 70-120 g/l, and more preferably 100±10 g/l. Preferably, the rHA is loaded in less than 0.5 column volumes, more preferably in less than 0.35 column volumes.

Suitably, the matrix comprises a boronic acid. The term “acid” as used herein includes the salts thereof. Advantageously, the boronic acid is bonded via a triazine or a substituted triazine, for example to form monoborotriazine or diborotriazine, to a support such as agarose. Preferably, the boronic acid is aminophenylboronic acid.

Publications that cover alternatives to phenylboronate, such as aliphatic and substituted aromatic ligands, include Adamek, V. et al (1992) J. Chrom. 625, 91-99, Singhal, R. P. et al (1991) J. Chrom. 543, 17-38 and Liu, X. et al (1994) 687, 61-69.

Suitably, following the affinity chromatography step the purified rHA solution is subjected to further purification, preferably further chromatographic purification. Preferably, the rHA is further purified using cation exchange chromatography and/or anion exchange chromatography. The order of the cation and anion exchange steps can be inter-changed while still performing their purification objectives. From an operational point of view, a better integrated process is cation exchange chromatography followed by anion exchange chromatography.

Suitably, the purified rHA solution produced according to the process described above undergoes one or more of: buffer exchange; concentration; dilution; dialysis; diafiltration; pH-adjustment (preferably to a pH greater than pH2.0 or pH4.0, and preferably to a pH less than pH10.0); treatment with a reducing agent (eg as described in EP 570 916); decolouration treatment (eg with charcoal); heating (including sterilisation); cooling or conditioning.

Second Process for Preparing Highly Purified rHA

Another process for purifying an rHA solution to a form suitable for use in the present invention comprises cation exchange chromatography and anion exchange chromatography, wherein the thus purified rHA solution optionally undergoes one or more of: buffer exchange; concentration; dilution; dialysis; diafiltration; pH adjustment (preferably to a pH greater than pH2.0 or pH4.0, and preferably to a pH less than pH10.0); addition of reducing agent; decolouration treatment (eg with charcoal); heating (including sterilisation); cooling; or conditioning.

The cation exchange chromatography step may follow the anion exchange chromatography step, or vice versa. Preferably, the cation exchange chromatography step is followed by the anion exchange chromatography step.

Preferably, between the anion and cation exchange steps there is no further purification step, although the rHA may be subjected to buffer exchange etc as noted above.

By conditioning, we mean any non-purifying handling step which improves the environment or condition of the rHA for the next step of the process or for final use. Conditioning can include the addition of an albumin stabiliser such as octanoate and/or other fatty acid, such as a C₆ or C₁₋₁₀ fatty acid, or sodium acetyl tryptophanate or mandelate. Conditioning can also include the addition of salts etc, and may involve adjusting the conductivity of the rHA solution.

The cation exchange step of the first and second aspects of the present invention may be run in negative or positive mode with respect to the rHA. In a preferred embodiment the cation exchange step is run in negative mode with respect to the rHA. Advantageously, the conditions are so chosen that glycosylated albumin binds more strongly to the cation exchange material than non-glycosylated albumin.

The cation exchange chromatography step of the first and second aspects of the present invention may utilise a commercial cation exchange matrix such as SP-Sepharose FF, SP-Spherosil, CM-Sepharose FF, CM-Cellulose, SE-Cellulose or S-Spheradex. Preferably, the cation exchange step utilises a matrix which comprises immobilised sulfopropyl substituents as cation exchangers.

Preferably, the rHA solution which undergoes cation exchange chromatography has a pH of 4.5-6.0, more preferably a pH of 5.0-5.6, and yet more preferably a pH of 5.2-5.4.

Preferably, the rHA solution which undergoes cation exchange chromatography has an rHA concentration of 10-250 g/l, preferably 20-70 g/l, and more preferably 50±10 g/l.

Preferably, the rHA solution which undergoes cation exchange chromatography has an octanoate ion concentration of 2-15 mM, preferably 5-10 mM, and more preferably 6-9 mM.

Conveniently, prior to the cation exchange step, the rHA solution undergoes one or more of the following processes: (i) pH-adjustment (preferably to a pH greater than pH2.0 or pH4.0, and preferably to a pH less than pH10.0); (ii) concentration; (iii) diafiltration; or (iv) conditioning by addition of a stabiliser such as octanoate and/or other fatty acid, such as a C₆ or C₁₀ fatty acid, or sodium acetyl tryptophanate or mandelate. Alternatively, or additionally, the rHA solution undergoes one or more of: buffer exchange; dilution; dialysis; diafiltration; treatment with a reducing agent; decolouration treatment (eg with charcoal); heating; cooling; or conditioning.

Generally, any modification involves additions, not removals. Preferably, the pH of the rHA solution is adjusted by the addition of acetic acid. Preferably, the rHA solution is concentrated by ultrafiltration.

The anion exchange chromatography step of the first and second aspects of the present invention may utilise a commercial anion exchange matrix such as Q Sepharose-FF, QMA-Spherosil, DEAE-Spherodex, Q-Hyper D, DEAE-cellulose, QAE-cellulose, or TMAE, DMAE, or DEAE Fractogel. Preferably, the anion exchange step utilises a matrix which comprises immobilised dialkylaminoalkyl (for example diethylaminoethyl) substituents as anion exchangers.

In one preferred embodiment the anion exchange chromatography step of the processes described above is run in negative mode with respect to the rHA.

Preferably, the rHA solution which undergoes negative mode anion exchange chromatography has a pH of 4.0-5.2, more preferably a pH of 4.2-4.9, and yet more preferably a pH of 4.5-4.7.

Preferably, the rHA solution which undergoes anion exchange chromatography has a conductivity of less than 4.0 mS/cm, and more preferably a conductivity of 1.0±0.5 mS/cm and yet more preferably 1.05±0.1 mS/cm.

Conveniently, prior to the anion exchange step, the rHA solution undergoes pH adjustment and/or dilution with water. Preferably, the pH of the rHA solution is adjusted with acetic acid.

In another preferred embodiment the anion exchange chromatography step of the processes described above is run in positive mode with respect to the rHA.

Suitably the rHA solution which undergoes positive mode anion exchange chromatography has a pH of 6.0-8.0, preferably a pH of 6.5-7.5, and yet more preferably a pH of 6.8 to 7.2. Preferably, the rHA solution has been pH-adjusted using orthophosphate ions.

In one preferred embodiment the rHA concentration is 10-100 g/l, more preferably 25-80 g/l, and most preferably 30-60 g/l. Preferably, the conductivity of the rHA solution is 1.0-2.0 mS/cm, preferably 1.2-1.6 mS/cm.

Suitably, the rHA is eluted from the anion exchanger with a buffer comprising 20-90 mM, preferably 30-70 mM and more preferably 35-65 mM of a phosphoric acid salt, for example sodium phosphate. Preferably, the rHA is eluted from the anion exchanger with a buffer of pH6.0-8.0, preferably pH6.5-7.5.

It is particularly preferred that the processes described above are preceded by one or more of the following steps: fermentation; primary separation; centrate conditioning; cation exchange chromatography, preferably using sulfopropyl substituents as cation exchangers; anion exchange chromatography, preferably using diethylaminoalkyl substituents as anion exchangers; or affinity chromatography, preferably using an affinity matrix which comprises an immobilised albumin-specific dye, preferably a Cibacron Blue type of dye.

A preferred process for purifying rHA comprises the following steps:
(a) subjecting an rHA solution to a cation exchange chromatography step run in positive mode with respect to the rHA;
(b) collecting an rHA-containing cation exchange eluate;
(c) subjecting the cation exchange eluate to an anion exchange chromatography step run in positive mode with respect to the rHA;
(d) collecting an rHA-containing anion exchange eluate;
(e) subjecting the anion exchange eluate to an affinity chromatography step run in positive mode with respect to the rHA;
(f) collecting an rHA-containing affinity chromatography eluate;
(g) subjecting the affinity chromatography eluate to an affinity chromatography step run in negative mode with respect to the rHA and in positive mode with respect to glycoconjugates (glycosylated albumin and/or glycoproteins);
(h) collecting the rHA-containing affinity chromatography flow-through;
(i) subjecting the affinity chromatography flow-through to a cation exchange chromatography step run in negative mode with respect to the rHA;
(j) collecting the rHA-containing cation exchange flow-through;
(k) subjecting the cation exchange flow-through to an anion exchange chromatography step run in negative mode or positive mode;
(l) collecting the rHA-containing anion exchange flow-through wherein the anion exchange step is run in negative mode; or cluting from the anion exchange matrix an anion exchange eluate wherein the anion exchange step is run in positive mode;
   and wherein any of the respective purification steps are optionally preceded or followed by one or more of: buffer exchange; concentration; dilution; dialysis; diafiltration; pH-adjustment (preferably to a pH greater than pH2.0 or pH4.0, and preferably to a pH less than pH 10.0); treatment with a reducing agent; decolouration treatment (eg with charcoal); heating (including sterilisation); cooling; or conditioning.

Accordingly, the purification steps may or may not be separated by one or more of: buffer exchange; concentration; dilution; dialysis; diafiltration; pH-adjustment; treatment with a reducing agent; decolouration treatment; heating; cooling; or conditioning.

When any step is run in the negative mode for rHA, washings may be collected as well as flow-through.

Another preferred process for purifying rHA comprises the following steps:
(a) subjecting an rHA solution to a cation exchange chromatography step run in positive mode with respect to the rHA;
(b) collecting an rHA-containing cation exchange eluate;
(c) subjecting the cation exchange eluate to an anion exchange chromatography step run in positive mode with respect to the rHA;
(d) collecting an rHA-containing anion exchange eluate;
(e) subjecting the anion exchange eluate to an affinity chromatography step run in positive mode with respect to the rHA;
(f) collecting an rHA-containing affinity chromatography eluate;
(g) subjecting the affinity chromatography eluate to an affinity chromatography step run in negative mode with respect to the rHA and in positive mode with respect to glycoconjugates;
(h) collecting the rHA-containing affinity chromatography flow-through;
(i) subjecting the affinity matrix flow-through to an anion exchange chromatography step run in negative or positive mode with respect to the rHA;
(j) collecting the rHA-containing anion exchange flow-through wherein the anion exchange step is run in negative mode; or eluting from the anion exchange matrix an anion exchange eluate wherein the anion exchange step is run in positive mode;
(k) subjecting the rHA solution purified by the anion exchange chromatography step to a cation exchange chromatography step run in negative mode with respect to the rHA;
(l) collecting the rHA-containing cation exchange flow-through;
   and wherein any of the respective purification steps are optionally preceded or followed by one or more of: buffer exchange; concentration; dilution; dialysis; diafiltration; pH-adjustment (preferably to a pH greater than pH2.0 or pH4.0, and preferably to a pH less than pH10.0); treatment with a reducing agent; decolouration treatment (eg with charcoal); heating (including sterilisation); cooling; or conditioning.

Accordingly, the purification steps may or may not be separated by: buffer exchange; concentration; dilution; dialysis; diafiltration; pH-adjustment; treatment with a reducing agent; decolouration treatment; heating; cooling; or conditioning.

Preferably, prior to the positive mode cation exchange step, the rHA solution is conditioned as above. Preferably, the octanoate is added thereto to a final concentration of from about 110 mM and the pH is adjusted to about 4.0-5.0.

Advantageously, the rHA retained in the positive cation exchange step is washed with a high salt solution (eg 0.5-3.0M NaCl buffered at pH3.5 to 4.5, preferably at about pH 4.0, with 10-100 mM, preferably 20-40 mM, for example 25-30 mM sodium acetate) before being eluted.

Preferably, the rHA is eluted in the cation exchange step using a buffer containing a compound having a specific affinity for albumin, especially an acid, for example octanoate or another fatty acid, for example C₆ or C₁₀.

Suitably, the rHA is eluted from the anion exchanger, of the first anion exchange step, with a buffer containing a high level (eg at least 50 mM, preferably 50-200 mM, for example 80-150 mM) of a boric acid salt, for example sodium or potassium tetraborate.

Preferably, the positive mode affinity chromatography step uses a resin comprising an immobilised albumin-specific dye, such as a Cibacron Blue type of dye, preferably immobilised on the resin via a spacer such as 1,4-diaminobutane or another spacer of C₁₋₈ preferably C₁₆, eg C₁₅ and most preferably C₄ length, preferably having α,ω-diamino substitution. Preferably, the matrix is the “Delta Blue Agarose” (DBA), prepared as described in WO 96/37515.

Third Process for Preparing Highly Purified rHA

Another process for purification of an rHA solution, particularly for reducing the level of nickel ions in an rHA solution, comprises subjecting the rHA solution to a pH of 2.5 to 7.5, preferably 2.5-6.0, and removing nickel ions. Preferably, the rHA solution is subjected to a pH of 4.0 to 7.5, preferably 4.0 to 6.0, more preferably pH4.0 to 5.5, yet more preferably pH4.0 to pH5.0, and most preferably to pH4.0 to 4.5.

Preferably, such a process comprises diafiltration against a buffer of pH2.5-6.0, or against a buffer having a pH within one of the aforementioned pH ranges. Alternatively, nickel removal can be achieved using gel permeation chromatography with a buffer having a pH within one of the above-listed pH ranges. Gel permeation chromatography may be performed using Sephacryl S200 HR. Preferably, the buffer comprises acetate and/or malate ions. Alternatively, there is sufficient buffering capacity from rHA to adjust the pH and perform diafiltration/gel permeation chromatography with water.

The nickel ions can alternatively be chelated and removed from the rHA. This can be achieved using a chelating agent such as iminodiacetic acid immobilised on Sepharose (Chelating Sepharose, Pharmacia) or another polymer (such as Chelex, Bio Rad Laboratories) at a low pH, preferably pH 4.0 to 6.0, more preferably pH4.0 to 4.5.

Preferably, when the product from the process just described is subjected immediately to negative cation exchange chromatography it is preferred that the process comprises subjecting the rHA solution to a pH of 5.0-5.6. Conversely, when the product from the process is not subjected immediately to negative anion exchange chromatography it is preferred that the process comprises subjecting the rHA solution to a pH of 4.3-4.9.

A purified rHA solution prepared as described above may be further processed. For example, it may be ultrafiltered through an ultrafiltration membrane to obtain an ultrafiltration retentate having an rHA concentration of at least about 10 g, preferably at least 40 g or more preferably about 80 g, rHA per litre, with the ultrafiltration retentate being diafiltered against at least 5 retentate equivalents of water.

As described above, highly purified rHA for use in accordance with the present invention may be obtained from an impure rHA solution. The process may comprise one or more of the following steps: culturing in a fermentation medium a micro-organism transformed with a nucleotide sequence encoding the amino acid sequence of human albumin; preferably separating the micro-organism from the fermentation medium; conditioning the medium, if necessary, for further purification; passing the conditioned medium through three successive chromatography steps; ultrafiltering/diafiltering the product; passing the ultrafiltered product through a further chromatography step; ultrafiltering/diafiltering again before purification through two further chromatographic steps; and final ultrafiltration/diafiltration.

Preceding or following any of the process steps described above, the rHA solution may undergo buffer exchange, concentration, dilution, heating (including sterilisation), cooling or may have salts etc added to the rHA solution which may, for example, condition or adjust the pH of the solution. Optionally, the rHA may be treated with a reducing agent or may undergo a decolouration step.

The invention will now be described in greater detail, by way of example only, with reference to the following Example, and the accompanying drawings, in which:

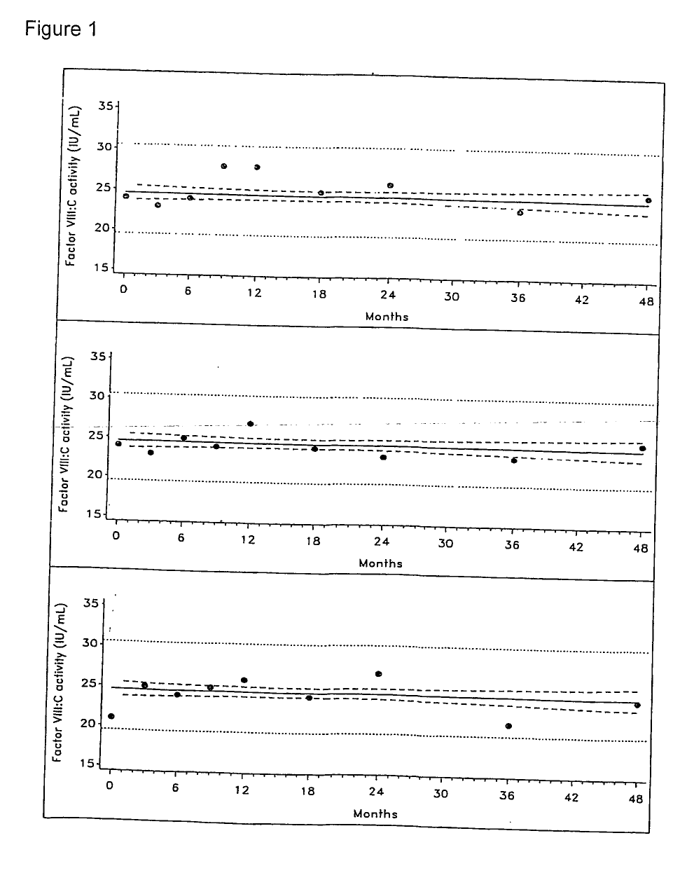

FIG. 1 shows the measured F-VIII:C activity for compositions (three lots) comprising F-VIII stabilised with 0.5% w/v highly purified rHA in a long-term stability study conducted over 48 months (fitted regression lines with one-sided 95% confidence limits as broken lines); and

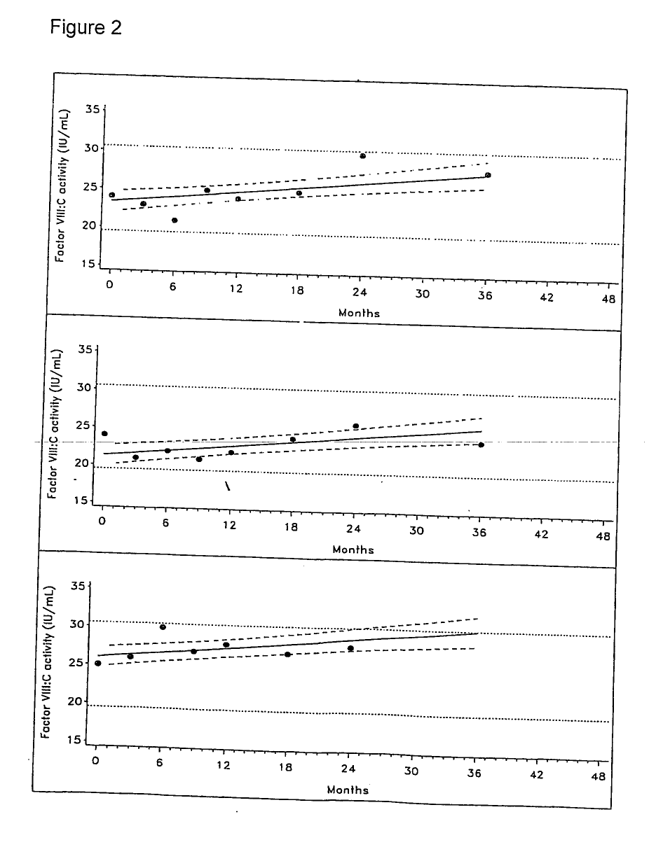

FIG. 2 shows corresponding data for compositions stabilised with serum-derived HSA.

### EXAMPLE 1

Investigational lots of an F-VIII preparation were prepared as follows:

Highly purified rHA was added to solutions of F-VIII and other excipients. The solutions were filled into vials containing 250 IU of F-VIII activity. The solutions were then lyophilised.

The samples were stored at 5° C., and reconstituted at intervals over a period of 48 months with water for injection. The reconstituted solutions had the following composition:

| | |
|---|---|
| Highly purified rHA | 5 mg/ml |
| Glycine | 20 mg/ml |
| Sodium citrate | 5.35 mg/ml |
| Sodium chloride | 3 mg/ml |

The following variables were investigated over a period of 48 months:
Residual moisture
Dissolution time
pH
Protein content
Factor VIII:C activity
vWF:RcoF activity
vWF antigen
vWF multimers
Polymers and aggregates

The data observed were compared with corresponding data obtained under identical conditions for corresponding samples in which the highly purified rHA was replaced by the same concentration of serum-derived HSA.

Results

No significant difference was observed between the behaviour of the samples containing highly purified rHA and those containing serum-derived HSA in respect of the following parameters:
Residual moisture
Dissolution time
pH
Protein content
vWF:RcoF activity
vWF antigen
vWF multimers
Polymers and aggregates

However, the Factor VIII:C activity of the two products showed a significant difference, as illustrated in FIGS. 1 and 2.

The observed change in Factor VIII:C activity over 36 months was an average decrease of 0.2 IU/ml for the samples comprising highly purified rHA (FIG. 1) and an average increase of 4.4 IU/ml for the samples comprising serum-derived HSA (FIG. 2). This difference was significant (p=0.004).

1. A composition comprising a non-albumin protein, the composition further comprising a highly purified rHA in an amount sufficient to stabilize the non-albumin protein.

2. A composition comprising a non-albumin protein, the composition further comprising highly purified rHA and one or more additional stabilizing agents.

3. A composition as claimed in claim 1, wherein the non-albumin protein is a recombinant protein.

4. A composition as claimed in claim 2, wherein the additional stabilizing agent (s) are selected from ionic salts, amino acids, sugars, detergents and polymers.

5. A composition as claimed in claim 4, which comprises an ionic salt selected from potassium chloride, sodium chloride and calcium chloride, at a level such that, following reconstitution of the composition with water, the concentration of chloride ion is in the range 0 to 2 mg/ml.

6. A composition as claimed in claim 2, which comprises an amino acid selected from one or more of histidine, lysine, glycine and arginine, at a level such that, following reconstitution of the composition with water, the concentration of amino acid(s) is from 0 to 100 mg/ml.

7. A composition as claimed in claim 2, which comprises a polyoxyethylene sorbitan ester, at a level such that, following reconstitution of the composition with water, the concentration of the polyoxyethylene sorbitan ester is less than 1 mg/ml.

8. A composition as claimed in claim 2, which comprises a polyethylene glycol, at a level such that, following reconstitution of the composition with water, the concentration of polyethylene glycol is in the range 0 to 10 mg/ml.

9. A composition as claimed in claim 8, wherein the polyethylene glycol has an average molecular weight of less than 10,000 daltons.

10. A composition as claimed in claim 2, which comprises a sugar selected from mannitol, sucrose, fructose, lactose and maltose, at a level such that, following reconstitution of the composition with water, the concentration of sugar is in the range 0 to 50 mg/ml.

11. A composition as claimed in claim 1, which is in the form of an aqueous solution or suspension.

12. A composition as claimed in claim 1, which is in the form of a lyophilized powder.

13. A composition as claimed in claim 12, which comprises, when reconstituted with water, from about 0.1 mg/ml up to about 20 mg/ml highly purified rHA.

14. A composition as claimed in claim 13, which comprises, when reconstituted with water, from about 0.1 mg/ml up to about 5 mg/ml highly purified rHA.

15. A composition as claimed in claim 1, wherein the non-albumin protein is F-VIII.

16. A composition as claimed in claim 15, wherein the F-VIII is rF-VIII.

17. A composition as claimed in claim 1, wherein the non-albumin protein is selected from the group consisting of all or part of an enzyme, an enzyme inhibitor, an antigen, an antibody, a hormone, a factor involved in the control of coagulation, an interferon, a cytokine of a growth factor and/or a factor involved in cell differentiation of a factor involved in the genesis/resorption of bone tissues, of a factor involved in cellular motility or migration, of a bactericidal or antifungal factor, of a chemotactic factor, of a cytostatic factor, of a plasma or interstitial adhesive molecule or proteins involved in the formation of extracellular matrices, or alternatively any peptide sequence which is an antagonist or agonist of molecular and/or intercellular interactions involved in the pathologies of the circulatory and interstitial compartments.

18. A composition as claimed in claim 1, wherein the highly purified rHA exhibits one or more of the following properties:
(i) extremely low levels of colorants;
(ii) extremely low levels of, or is essentially free of, aluminum, lactate, citrate, metals, non-albumin human proteins, prokaryotic proteins, fragments of albumin, albumin aggregates or polymers, or endotoxin, bilirubin, heme, yeast proteins, animal proteins and viruses;
(iii) at least 99.5% monomeric and dimeric;
(iv) a nickel ion level of less than 100 ng, based on one gram of albumin;
(v) a glycation level of less than 0.6 moles hexose/mole protein as measured in the Amadori product assay;
(vi) at least 90% of the albumin molecules have an intact C-terminus;
(vii) a content of concanavalin A-binding albumin of less than 0.5% (w/w);
(viii) a free thiol content of at least 0.85 mole SH/mole protein when measured by using Ellman's Reagent;
(ix) substantially no C18 or C20 fatty acids, when analyzed by acidic solvent extraction and gas chromatography of free fatty acids using a C17:0 internal standard; and
(x) a high degree of molecular weight homogenity, with a molecular weight distribution of at least 50 percent of albumin molecules with a molecular weight spread no greater than 2000 daltons when determined by mass analysis using electrospray mass spectrometry.

19. A composition as claimed in claim 1, wherein the highly purified rHA is characterized by the following combination of characteristics:
(i) a molecular weight distribution of at least 90 percent, of albumin molecules with a molecular weight spread no greater than 1000 daltons when determined by mass analysis using electrospray mass spectrometry;
(ii) a glycation level of less than 0.10 moles hexose/mole protein; and
(iii) a content of concanavalin A-binding albumin of less than 0.3% (w/w).

20. A composition as claimed in claim 1, wherein the highly purified rHA is prepared by subjecting a first rHA solution of pH8.0-9.5, and having a conductivity in the range of 1 to 75 mS·cm⁻¹, to an affinity chromatography step which is run in negative mode with respect to the rHA and which utilizes an affinity matrix comprising immobilized dihydroxyboryl groups, thereby obtaining a purified rHA solution.

21. A composition as claimed in claim 1, wherein the highly purified rHA is prepared by subjecting an rHA solution to cation exchange chromatography and anion exchange chromatography, wherein the thus purified rHA solution optionally undergoes one or more of: buffer exchange; concentration; dilution; dialysis; diafiltration; pH adjustment addition of reducing agent; decoloration treatment; heating; cooling; or conditioning.

22. A composition as claimed in claim 1, wherein the highly purified rHA is prepared by a process comprising the following steps:
(a) subjecting an rHA solution to a cation exchange chromatography step run in positive mode with respect to the rHA;
(b) collecting an rHA-containing cation exchange eluate;
(c) subjecting the cation exchange eluate to an anion exchange chromatography step run in positive mode with respect to the rHA;
(d) collecting an rHA-containing anion exchange eluate;
(e) subjecting the anion exchange eluate to an affinity chromatography step run in positive mode with respect to the rHA;
(f) collecting an rHA-containing affinity chromatography eluate;
(g) subjecting the affinity chromatography eluate to an affinity chromatography step run in negative mode with respect to the rHA and in positive mode with respect to glycoconjugates;
(h) collecting the rHA-containing affinity chromatography flow-through;
(i) subjecting the affinity chromatography flow-through to a cation exchange chromatography step run in negative mode with respect to the rHA;
(j) collecting the rHA-containing cation exchange flow-through;
(k) subjecting the cation exchange flow-through to an anion exchange chromatography step run in negative mode or positive mode;
(l) collecting the rHA-containing anion exchange flow-through wherein the anion exchange step is run in negative mode; or eluting from the anion exchange matrix an anion exchange eluate wherein the anion exchange step is run in positive mode;
and wherein any of the respective purification steps are optionally preceded or followed by one or more of: buffer exchange; concentration; dilution; dialysis; diafiltration; pH-adjustment; treatment with a reducing agent; decoloration treatment; heating; cooling; or conditioning.

23. A composition as claimed in claim 1, wherein the highly purified rHA is prepared by a process comprising the following steps:
(a) subjecting an rHA solution to a cation exchange chromatography step run in positive mode with respect to the rHA;
(b) collecting an rHA-containing cation exchange eluate;
(c) subjecting the cation exchange eluate to an anion exchange chromatography step run in positive mode with respect to the rHA;
(d) collecting an rHA-containing anion exchange eluate;
(e) subjecting the anion exchange eluate to an affinity chromatography step run in positive mode with respect to the rHA;
(f) collecting an rHA-containing affinity chromatography eluate;
(g) subjecting the affinity chromatography eluate to an affinity chromatography step run in negative mode with respect to the rHA and in positive mode with respect glycoconjugates;
(h) collecting the rHA-containing affinity chromatography flow-through;
(i) subjecting the affinity matrix flow-through to an anion exchange chromatography step run in negative or positive mode with respect to the rHA;
(j) collecting the rHA-containing anion exchange flow-through wherein the anion exchange step is run in negative mode; or eluting from the anion exchange matrix an anion exchange eluate wherein the anion exchange step is run in positive mode;
(k) subjecting the rHA solution purified by the anion exchange chromatography step to a cation exchange chromatography step run in negative mode with respect to the rHA;
(l) collecting the rHA-containing cation exchange flow-through;
and wherein any of the respective purification steps are optionally preceded or followed by one or more of: buffer exchange; concentration; dilution; dialysis; diafiltration; pH-adjustment; treatment with a reducing agent; decoloration treatment; heating; cooling; or conditioning.

24. A composition as claimed in claim 1, wherein the highly purified rHA is prepared by subjecting an rHA solution to a pH of 2.5 to 7.5, and removing nickel ions.

25. A process for the preparation of a composition comprising a non-albumin recombinant protein, which process comprises the steps of
a) causing a cell transformed with a nucleotide sequence coding the non-albumin recombinant protein to express the non-albumin recombinant protein;
b) isolating and/or purifying the non-albumin recombinant protein; wherein step a) and/or step b) is carried out in the presence of a first form of rHA, which first form of rHA is less pure than a second form of rHA;
c) separating the isolated and/or purified non-albumin protein obtained in step b) from the first form of rHA; and
d) combining the isolated and/or purified non-albumin recombinant protein with the second form of rHA and optionally with other excipients in order to provide a stable composition.

26. A method for preserving or maintaining the F-VIII activity of a composition comprising F-VIII, which method comprises adding to the composition a stabilizing amount of highly purified rHA.

27. A method as claimed in claim 26, wherein the F-VIII is rF-VIII.

28. A composition as claimed in claim 2 wherein the non-albumin protein is a recombinant protein.

29. A composition as claimed in claim 2, which is in the form of an aqueous solution or suspension.

30. A composition as claimed in claim 2, which is in the form of a lyophilized powder.

31. A composition as claimed in claim 30, which comprises, when reconstituted with water, from about 0.1 mg/ml up to about 20 mg/ml highly purified rHA.

32. A composition as claimed in claim 31, which comprises, when reconstituted with water, from about 0.1 mg/ml up to about 5 mg/ml highly purified rHA.

33. A composition as claimed in claim 2, wherein the non-albumin protein is F-VIII.

34. A composition as claimed in claim 33, wherein the F-VIII is rF-VIII.

35. A composition as claimed in claim 2, wherein the non-albumin protein is selected from the group consisting of all or part of an enzyme, an enzyme inhibitor, an antigen, an antibody, a hormone, a factor involved in the control of coagulation, an interferon, a cytokine of a growth factor and/or a factor involved in cell differentiation, of a factor involved in the genesis/resorption of bone tissues, of a factor involved in cellular motility or migration, of a bactericidal or antifungal factor, of a chemotactic factor, of a cytostatic factor, of a plasma or interstitial adhesive molecule or proteins involved in the formation of extracellular matrices, or alternatively any peptide sequence which is an antagonist or agonist of molecular and/or intercellular interactions involved in the pathologies of the circulatory and interstitial compartments.

36. A composition as claimed in claim 2, wherein the highly purified rHA exhibits one or more of the following properties:
(i) extremely low levels of colorants;
(ii) extremely low levels of, or is essentially free of, aluminum, lactate, citrate, metals, non-albumin human proteins, prokaryotic proteins, fragments of albumin, albumin aggregates or polymers, or endotoxin, bilirubin, heme, yeast proteins, animal proteins and viruses;
(iii) at least 99.5% monomeric and dimeric;
(iv) a nickel ion level of less than 100 ng, based on one gram of albumin;
(v) a glycation level of less than 0.6 moles hexose/mole protein as measured in the Amadori product assay;
(vi) at least 90% of the albumin molecules have an intact C-terminus;
(vii) a content of concanavalin A-binding albumin of less than 0.5% (w/w);
(viii) a free thiol content of at least 0.85 mole SH/mole protein when measured by using Ellman's Reagent;
(ix) substantially no C18 or C20 fatty acids, when analyzed by acidic solvent extraction and gas chromatography of free fatty acids using a C17:0 internal standard; and
(x) a high degree of molecular weight homogenity with a molecular weight distribution of at least 50 percent of albumin molecules with a molecular weight spread no greater than 2000 daltons when determined by mass analysis using electrospray mass spectrometry.

37. A composition as claimed in claim 2, wherein the highly purified rHA is characterized by the following combination of characteristics:
(i) a molecular weight distribution of at least 90 percent of albumin molecules with a molecular weight spread no greater than 1000 daltons when determined by mass analysis using electrospray mass spectrometry;
(ii) a glycation level of less than 0.10 moles hexose/mole protein; and
(iii) a content of concanavalin A-binding albumin of less than 0.3% (w/w).

38. A composition as claimed in claim 2, wherein the highly purified rHA is prepared by subjecting a first rHA solution of pH8.0-9.5, and having a conductivity in the range of 1 to 75 mS·cm⁻¹, to an affinity chromatography step which is run in negative mode with respect to the rHA and which utilizes an affinity matrix comprising immobilized dihydroxyboryl groups, thereby obtaining a purified rHA solution.

39. A composition as claimed in claim 2, wherein the highly purified rHA is prepared by subjecting an rHA solution to cation exchange chromatography and anion exchange chromatography, wherein the thus purified rHA solution optionally undergoes one or more of: buffer exchange; concentration; dilution; dialysis; diafiltration; ph adjustment addition of reducing agent; decoloration treatment; heating; cooling; or conditioning.

40. A composition as claimed in claim 2, wherein the highly purified rHA is prepared by a process comprising the following steps:
(a) subjecting an rHA solution to a cation exchange chromatography step run in positive mode with respect to the rHA;
(b) collecting an rHA-containing cation exchange eluate;
(c) subjecting the cation exchange eluate to an anion exchange chromatography step run in positive mode with respect to the rHA;
(d) collecting an rHA-containing anion exchange eluate;
(e) subjecting the anion exchange eluate to an affinity chromatography step run in positive mode with respect to the rHA;
(f) collecting an rHA-containing affinity chromatography eluate;
(g) subjecting the affinity chromatography eluate to an affinity chromatography step run in negative mode with respect to the rHA and in positive mode with respect to glycoconjugates;
(h) collecting the rHA-containing affinity chromatography flow-through;
(i) subjecting the affinity chromatography flow-through to a cation exchange chromatography step run in negative mode with respect to the rHA;
(j) collecting the rHA-containing cation exchange flow-through;
(k) subjecting the cation exchange flow-through to an anion exchange chromatography step run in negative mode or positive mode;
(l) collecting the rHA-containing anion exchange flow-through wherein the anion exchange step is run in negative mode; or eluting from the anion exchange matrix an anion exchange eluate wherein the anion exchange step is run in positive mode;
and wherein any of the respective purification steps are optionally preceded or followed by one or more of: buffer exchange; concentration; dilution; dialysis; diafiltration; pH-adjustment; treatment with a reducing agent; decoloration treatment; heating; cooling; or conditioning.

41. A composition as claimed in claim 2, wherein the highly purified rHA is prepared by a process comprising the following steps:
(a) subjecting an rHA solution to a cation exchange chromatography step run in positive mode with respect to the rHA;
(b) collecting an rHA-containing cation exchange eluate;
(c) subjecting the cation exchange eluate to an anion exchange chromatography step run in positive mode with respect to the rHA;
(d) collecting an rHA-containing anion exchange eluate;
(e) subjecting the anion exchange eluate to an affinity chromatography step run in positive mode with respect to the rHA;
(f) collecting an rHA-containing affinity chromatography eluate;
(g) subjecting the affinity chromatography eluate to an affinity chromatography step run in negative mode with respect to the rHA and in positive mode with respect to glycoconjugates;
(h) collecting the rHA-containing affinity chromatography flow-through;
(i) subjecting the affinity matrix flow-through to an anion exchange chromatography step run in negative or positive mode with respect to the rHA;
(j) collecting the rHA-containing anion exchange flow-through wherein the anion exchange step is run in negative mode; or eluting from the anion exchange matrix an anion exchange eluate wherein the anion exchange step is run in positive mode;
(k) subjecting the rHA solution purified by the anion exchange chromatography step to a cation exchange chromatography step run in negative mode with respect to the rHA;
(l) collecting the rHA-containing cation exchange flow-through;
and wherein any of the respective purification steps are optionally preceded or followed by one or more of: buffer exchange; concentration; dilution; dialysis; diafiltration; pH-adjustment; treatment with a reducing agent; decoloration treatment; heating; cooling; or conditioning.

42. A composition as claimed in claim 2, wherein the highly purified rHA is prepared by subjecting an rHA solution to a pH of 2.5 to 7.5, and removing nickel ions.
